(19) United States
(12) Patent Application Publication
(10) Pub. No.: US 2010/0189804 A1
(43) Pub. Date: Jul. 29, 2010

(54) ANTIMICROBIAL CARBON BLACK DISPERSION

(75) Inventors: David Schlossman, Short Hills, NJ (US); Yun Shao, Belle Mead, NJ (US); Carl Orr, Scotch Plains, NJ (US); Robert Eskay, Edison, NJ (US)

Correspondence Address: HANDAL & MOROFSKY, LLC, 501 KINGS HIGHWAY EAST, Fairfield, CT, 06825-4867, US

(73) Assignee: Kobo Products, Inc., South Plainfield, NJ (US)

(21) Appl. No.: 12/358,491

(22) Filed: Jan. 23, 2009

(51) Int. Cl. A61K 8/19; A61Q 1/00; A61P 31/04

(52) U.S. Cl. 424/600

(57) ABSTRACT

Disclosed is an antimicrobial carbon black dispersion which exhibits excellent dispersant properties and also inhibits microbial contamination when used in consumer products such as cosmetic formulations. Also disclosed are methods for producing and using the antimicrobial carbon black dispersion.

### FIELD OF THE INVENTION

The present invention relates generally to carbon black dispersions and, more particularly, to aqueous carbon black dispersions having antimicrobial properties.

### BACKGROUND

It is known that special color effects can be achieved by incorporation of carbon black into pigments. Among conventional black pigments, carbon black exhibits excellent blackness. Carbon black has long been used generally for paints, printing inks, cosmetics, rubbers, resin compositions and the like. For many applications, pigments have an advantage over dyes inasmuch as their resistance to light is very high and they are water-resistant.

Carbon black is a powder consisting of fine amorphous particles having an average primary particle size as small as 0.05 to 0.5 nm. Due to the fine primary particle size, carbon black particles tend to heavily aggregate and can be difficult to disperse uniformly in vehicles or resin compositions. This compromises the performance of the carbon black as a black pigment in product formulation.

In addition, fine carbon black particles have a very low bulk density, typically about 0.1 g/cm³ and can, accordingly, easily become airborne and contaminate work environments. Therefore, handling of carbon black powder typically is avoided by many end users.

The production of aqueous carbon black dispersions is known (see, for example, U.S. Pat. Nos. 6,685,769, 6,171,382 and 5,538,548, all of which are incorporated by reference herein). However, known aqueous carbon black dispersions used in consumer product formulations, such as cosmetic formulations, can have the disadvantage of being prone to microbial contamination.

Thus, there exists a need to produce a carbon black dispersion that has excellent dispersant properties as well as the ability to inhibit microbial contamination. Such a carbon black dispersion can then be employed with a higher degree of efficacy and safety in consumer product formulations.

### SUMMARY OF THE INVENTION

The present invention meets this need by providing an antimicrobial carbon black dispersion and methods of production and use of the antimicrobial carbon black dispersion.

In one aspect the present invention provides an antimicrobial carbon black dispersion, comprising carbon black, at least one dispersant, at least one alcohol and the balance water.

In another aspect, the present invention provides an antimicrobial carbon black dispersion, comprising about 20% to about 50% by weight carbon black; about 2% to about 40% by weight cationic block copolymer, about 2% to about 40% by weight nonionic surfactant, at least about 10% polyhydric alcohol, and optionally about 1% to about 20% by weight hydrophilic nonionic polymer, and about 30% to about 70% water.

In another aspect, the present invention provides a method of producing an antimicrobial carbon black dispersion, comprising feeding into a dispersal apparatus a composition comprising carbon black, at least one dispersant, at least one alcohol and the balance water.

In another aspect, the present invention provides a method of producing the antimicrobial carbon black dispersion, comprising feeding into a dispersal apparatus a composition comprising about 20% to about 50% by weight carbon black; about 2% to about 40% by weight cationic block copolymer, about 2% to about 40% by weight nonionic surfactant, at least about 10% polyhydric alcohol, optionally about 1% to about 20% by weight hydrophilic nonionic polymer, and about 30% to about 70% water; and using the dispersal apparatus to produce the antimicrobial carbon black dispersion from the composition.

In another aspect, the present invention provides a method of using an antimicrobial carbon black dispersion, comprising producing consumer product formulations such as, without limitation, cosmetic products, containing the antimicrobial carbon black dispersion of the present invention.

### DETAILED DESCRIPTION

In accordance with the invention, antimicrobial carbon black dispersions and methods of production and use of the antimicrobial carbon black dispersions are provided.

As used herein, “carbon black” refers to a particulate, mostly carbon material which may be produced, for example, by the incomplete combustion of heavy petroleum products, such as FCC tar, coal tar, and ethylene cracking tar. Carbon black is a form of amorphous carbon that has a high surface area to volume ratio, and as such it is one of the first nanomaterials to find common usage.

As used herein, “antimicrobial” refers to a substance that kills or inhibits the growth of microbes such as bacteria, fungi, or viruses.

As used herein, a “dispersion” refers to a liquid composition in which there is largely an even dissemination of one or more particulate substances in a liquid.

As used herein, a “dispersant” and is an agent that allows for one or more particulate substances to largely disseminate in a liquid.

In one embodiment of the present invention, there is provided an antimicrobial carbon black dispersion, comprising carbon black, at least one dispersant, at least one alcohol and the balance water.

In another embodiment of the present invention, there is provided an antimicrobial carbon black dispersion, comprising about 20% to about 50% by weight carbon black; about 2% to about 40% by weight cationic block copolymer, about 2% to about 40% by weight nonionic surfactant, at least about 10% polyhydric alcohol, optionally about 1% to about 20% by weight hydrophilic nonionic polymer, and about 30% to about 70% water.

In an exemplary embodiment, the antimicrobial carbon black dispersion comprises about 25% by weight carbon black; about 3% by weight cationic block copolymer, about 3% by weight nonionic surfactant; about 11% by weight polyhydric alcohol; about 2% hydrophilic nonionic polymer, and about 56% water.

In another embodiment of the present invention, there is provided a method of producing an antimicrobial carbon black dispersion, comprising feeding into a dispersal apparatus a composition comprising carbon black, at least one dispersant, at least one alcohol and the balance water.

In another embodiment of the present invention, there is provided a method of producing the antimicrobial carbon black dispersion, comprising feeding into a dispersal apparatus a composition comprising about 20% to about 50% by weight carbon black, about 2% to about 40% by weight cationic block copolymer, about 2% to about 40% by weight nonionic surfactant, at least about 10% polyhydric alcohol, optionally about 1% to about 20% by weight hydrophilic nonionic polymer, and about 30% to about 70% water; and using the dispersal apparatus to produce the antimicrobial carbon black dispersion from the composition.

In an exemplary embodiment, the composition that is fed into the dispersal apparatus comprises about 25% by weight carbon black; about 3% by weight cationic block copolymer, about 3% nonionic surfactant; about 11% by weight polyhydric alcohol; about 2% hydrophilic nonionic polymer, and about 56% by weight water.

Suitable dispersal apparatuses are known by those skilled in the art and include, without limitation, high speed mixers, media mills, bead mills, ultrasonic mills and ultra-turrax for dispersal of the carbon black.

In an exemplary embodiment, the dispersal apparatus is a media mill, which is a dry airswept apparatus employed for the production of fine powders having diameters typically below 45 μm.

In another embodiment of the present invention, there is provided a method of using an antimicrobial carbon black dispersion, comprising producing consumer product formulations, such as, without limitation, cosmetic products, containing the antimicrobial carbon black dispersion of the present invention. It is within the purview of those skilled in the art to know how to formulate the antimicrobial carbon black dispersion of the present invention into various consumer products, such as cosmetic formulations. Prior art techniques of employing aqueous dispersions of particles may generally be employed.

The weight ratio of cationic block copolymer to nonionic surfactant in the carbon black dispersion is between 1:5 and 5:1. Known cationic and nonionic surfactant may be employed in the present invention and are known by those skilled in the art.

Suitable cationic wetting agents that can be used as surfactants in the present invention include, without limitation, cationic block copolymers such as alkyl acrylate (where the alkyl group contains about 1 to about 4 carbon atoms) and quaternized aminoalkyl acrylate.

Suitable nonionic wetting agents that can be used as surfactants in the present invention include, without limitation, acetylated lanolin alcohol, polysorbated 80, cetyl acetate, polyoxyethylene acrylic acid, amine oxides, alkanolamides, derivatives of carbohydrates, ethylene oxide/propylene oxide copolymers, ethoxylates of fatty acids, sorbitan derivatives, ethylene glycol esters, propylene glycol esters, glycerine esters and polyglycerine esters, alkylamines and alkyl-imidazolines and combinations thereof.

In an exemplary embodiment, the nonionic surfactants that can be used in the present invention include acetylated lanolin alcohol, polysorbate 80 or cetyl acetate.

Suitable hydrophilic nonionic wetting agents that can be used as surfactants in the present invention include, without limitation, poly(N-vinylpyrrolidone), quaternary ammonium compounds, amine salts, imidazolium salts and combinations thereof.

In an exemplary embodiment, a possible hydrophilic nonionic surfactant that can be used in the present invention is poly(N-vinylpyrrolidone) (PVP).

In addition to cationic and nonionic wetting agents, anionic wettings agents, known by those skilled in the art, may be used in the formulation of the antimicrobial carbon black dispersion of the present invention.

The antimicrobial action of the carbon black dispersion of the invention is provided by the one or more polyhydric alcohols that are contained in the formulation of the carbon black dispersion.

Suitable polyhydric alcohols that can be used in the present invention include, without limitation, butylene glycol, caprylyl glycol propylene glycol, dipropylene glycol, tripropylene glycol, propylene carbonate and combinations thereof. In an exemplary embodiment, the polyhydric alcohols that can be used in the present invention is a mixture of butylene glycol and caprylyl glycol. In another exemplary embodiment, the polyhydric alcohol is butylene glycol. In addition, alcohols other than polyhydric alcohols known by those skilled in the art may be used in the formulation of the antimicrobial carbon black dispersion of the present invention.

Carbon black having a specific surface area of about 100 to about 400 m²/gram can be used in the present invention.

In an exemplary embodiment, the specific surface area of carbon black is about 200 to about 260 m²/gram.

The pH of the carbon black dispersion of the present invention is in the basic range of about 8-10.

The carbon black dispersion of the present invention is excellently suited for use as a black pigment in consumer product formulations such as cosmetic formulations, because of its characteristics of having substantially no agglomerated carbon black particles and its ability to substantially inhibit microbial contamination. Bacterial organisms that are susceptible to the antimicrobial action of the carbon black dispersion of the present invention include, without limitation, Staphylococcus aureus, Streptococcus agalactiae, Streptococcus faecalis, Eschericia coli, Proteus vulgaris, Mycobacterium smegmatis, Klebsiella pneumonia, Pseudomonas aeruginosa, Pseudomonas fluorescans, Pseudomonas oleovorans, Corynebacterium xerosis, Micrococus Iuteus, Bacillus cereus, Ravobacter suaveolens, Enterobacter aerogenes, Serratia marcescens, Shigella sonnei, Salmonella typhosa and Aspergillus niger.

Fungal organisms that are susceptible to the antimicrobial action of the carbon black dispersion of the present invention include, without limitation, Candida albicans, Candida parapsilosis, Chaetomium globosum, Cladosporium resinae, Gliocladium fimbriatum and Penicillium notatum.

The present invention is more particularly described in the following non-limiting examples, which are intended to be illustrative only, as numerous modifications and variations therein will be apparent to those skilled in the art.

### EXAMPLES

### Example 1

### Inhibition of Microbial Growth

1. Methodology

A study was undertaken to determine the effectiveness of three different carbon black dispersion compositions containing butylene glycol to inhibit bacterial and fungal growth. Methods for isolating and identifying microbial growth are well known by those skilled in the art and may generally be employed in the present invention.

Dispersions were prepared by putting all ingredients in a Hochmeyer dispersion mill (Hockmeyer Lab Mixer, Model 2HL) and mixing at 200 RPM using a dispersion blade.

The three different carbon black dispersions contained the following components:

Sample 1: 25% by weight carbon black; 0.5% by weight sodium lignin sulfonate; 0.5% by weight poly(N-vinylpyrrolidone) (PVP); 4% acetylated lanolin alcohol; 5% by weight butylene glycol; and the balance water.

Sample 2: 25% by weight carbon black; 0.5% by weight sodium lignin sulfonate; 0.5% by weight poly(N-vinylpyrrolidone) (PVP); 4% acetylated lanolin alcohol; 5% by weight butylene glycol; 1% by weight phenoxyatol; and the balance water.

Sample 3: 25% by weight carbon black; 0.5% by weight sodium lignin sulfonate; 0.5% by weight poly(N-vinylpyrrolidone) (PVP); 4% acetylated lanolin alcohol; 10% by weight butylene glycol; and the balance water.

2. Results

TABLE 1
Butylene Glycol Inhibition of Microbial
Growth

| Sample Number | Results |
|---|---|
| 1 | 10 microbial; |
| | No gram negative bacterial |
| | growth; |
| | 10 yeast/mold |
| 2 | <10 microbial; |
| | No gram negative bacterial |
| | growth; |
| | 45 yeast/mold |
| 3 | <10 cfu/g; |
| | No gram negative bacterial |
| | growth |

As shown in Table 1, Sample 1 having 5% by weight butylene glycol inhibited bacterial growth to 10 microbial. All gram negative bacteria were inhibited. Fungal growth was inhibited to 10 yeast/mold. Sample 2 having 5% by weight butylene glycol and 1% phenoxytol inhibited bacterial growth to less than 10 microbial and all gram negative bacteria were inhibited. Fungal growth was inhibited to 45 yeast/mold. Sample 3 having 10% butylene glycol inhibited bacterial growth to <10 cfu/g and all gram negative bacteria were inhibited.

### Example 2

### Production of a Carbon Black Dispersion

A 4 kg batch of a carbon black dispersion composition was produced using the ingredients shown in Table 2.

TABLE 2
Formula of Pre-Mix Dispersion

| | RAW | AMOUNT | AMOUNT | | CHEMICAL |
|---|---|---|---|---|---|
| | MATERIALS | (kg) | (%) | FUNCTION | NAME |
| 1 | Distilled H₂O | 2.240 | 56 | | Water |
| 2 | Butylene | 0.400 | 10 | Polyhydric alcohol | Polyhydric alcohol |
| | Glycol | | | | |
| 3 | Caprylyl | 0.040 | 1 | Polyhydric alcohol | Polyhydric alcohol |
| | Glycol | | | | |
| 4 | EFKA-4585 | 0.120 | 3 | Cationic block | Alkyl acrylate and |
| | | | | copolymer | quaternized |
| | | | | | dialkylaminoalkyl |
| | | | | | acrylate |
| 5 | Solulan 98 | 0.120 | 3 | Nonionic dispersant | Acetylated lanolin |
| | | | | | alcohol |
| | | | | | Polysorbate 80 |
| | | | | | Cetyl acetate |
| 6 | Unipure Black | 1 | 25 | | Carbon Black |
| | LC902 | | | | |
| 7 | PVP-K30 | 0.080 | 2 | Hydrophilic nonionic | poly(N- |
| | | | | polymer | vinylpyrrolidone) |
| Total | | 4.00 | 100 | | (PVP) |

Ingredients 1 through 5, distilled water, butylene glycol, caprylyl glycol, EFKA-4585 (Ciba Specialty Chemicals Heerenveen b.v., The Netherlands) and Solulan 98 (Lubrizol Advanced Materials, Inc., OH) were added to a 5 gallon pail and mixed to formula uniform solution, Ingredient 6, Unipure carbon black Unipure, LCW, USA, NJ) was then added slowly under mixing. A Hockmeyer dispersion mill (Hockmeyer Lab Mixer, Model 2HL) with a dispersing blade was used and operated at a speed of 200 RPM. Speeds of 100-300 rpm were also found to be effective. When all six ingredients were mixed together, ingredient 7, the hydrophilic nonionic polymer, PVP-K30 (ISP Technologies, Inc., CT) was added under mixing at 200 RPM. Mixing was stopped when the dispersion appeared smooth. When the dispersion was tested on a Hegman gauge, the dispersion passed a fineness of 8 and was pourable as is or with slight agitation.

The carbon black dispersions of the above examples may be used as an ingredient in the formulation of various cosmetics using existing recipes, formulas and techniques which are currently employed in the manufacture of cosmetics, such as mascara, eye shadow, foundation, sun tanning lotion, bronzer, lipstick, nail polish, and so forth. It is also understood that this dispersion may be incorporated into various emulsions such as oil in water emulsions, water in oil emulsions, and in aqueous and other types of products. Likewise, the carbon black dispersions of the present invention may be incorporated into nontraditional chemistries, such as silicone fluid-based chemistries which may or may not be analogous to oil-based products.

While illustrative embodiments have been described above, it is, of course, understood that various modifications will be apparent to those of ordinary skill in the art. Many such modifications are contemplated as being within the spirit and scope of the following claims.

What is claimed is:

1. An antimicrobial carbon black dispersion, comprising carbon black, at least one dispersant, at least one alcohol and the balance water.

2. The antimicrobial carbon black dispersion of claim 1, wherein the at least one dispersant is selected from the group consisting of cationic dispersants, nonionic dispersants and anionic dispersants.

3. The antimicrobial carbon black dispersion of claim 1, wherein the at least one alcohol is a polyhydric alcohol.

4. The antimicrobial carbon black dispersion of claim 3, wherein the polyhydric alcohol is selected from the group consisting of butylene glycol and caprylyl alcohol.

5. An antimicrobial carbon black dispersion as in claim 1, comprising about 20% to about 50% by weight carbon black, about 2% to about 40% by weight cationic block copolymer, about 2% to about 40% by weight nonionic surfactant, at least about 10% polyhydric alcohol, optionally about 1% to about 20% by weight hydrophilic nonionic polymer, and about 30% to about 70% water.

6. The antimicrobial carbon black dispersion of claim 5, wherein the dispersion comprises about 25% by weight carbon black, about 3% by weight cationic block copolymer, about 3% by weight nonionic surfactant, about 11% by weight polyhydric alcohol, about 2% hydrophilic nonionic polymer, and about 56% water.

7. The antimicrobial carbon black dispersion of claim 5, wherein the cationic block copolymer is alkyl acrylate and quaternized dialkylaminoalkyl acrylate.

8. The antimicrobial carbon black dispersion of claim 5, wherein the nonionic surfactant is selected from the group consisting of acetylated lanolin alcohol, polysorbated 80, cetyl acetate, polyoxyethylene acrylic acid, amine oxides, alkanolamides, derivatives of carbohydrates, ethylene oxide/propylene oxide copolymers, ethoxylates of fatty acids, sorbitan derivatives, ethylene glycol esters, propylene glycolesters, glycerine esters and polyglycerine esters, alkylamines and alkyl-imidazolines, and combinations thereof.

9. The antimicrobial carbon black dispersion of claim 8, wherein the nonionic surfactant is selected from the group consisting of acetylated lanolin alcohol, polysorbate 80 and cetyl acetate.

10. The antimicrobial carbon black dispersion of claim 5, wherein the hydrophilic nonionic polymer is selected from the group consisting of poly(N-vinylpyrrolidone), quaternary ammonium compounds, amine salts, imidazolium salts and combinations thereof.

11. The antimicrobial carbon black dispersion of claim 10, wherein the hydrophilic nonionic polymer is poly(N-vinylpyrrolidone).

12. The antimicrobial carbon black dispersion of claim 5, wherein the polyhydric alcohol is selected from the group consisting of butylene glycol, caprylyl glycol, propylene glycol, dipropylene glycol, tripropylene glycol, propylene carbonate and combinations thereof.

13. The antimicrobial carbon black dispersion of claim 12, wherein the polyhydric alcohol is a mixture of butylene glycol and caprylyl glycol.

14. The antimicrobial carbon black dispersion of claim 13, wherein the polyhydric alcohol is butylene glycol.

15. The antimicrobial carbon black dispersion of claim 5, wherein the ratio of cationic block copolymer to nonionic surfactant is between about 1:5 and 5:1.

16. The antimicrobial carbon black dispersion of claim 5, wherein the specific surface area of the carbon black is between about 100 to 400 m²/gram.

17. The antimicrobial carbon black dispersion of claim 16, wherein the specific surface area of the carbon black is between about 200 to 260 m²/gram.

18. A method of producing an antimicrobial carbon black dispersion, comprising feeding into a dispersal apparatus a composition comprising carbon black, at least one dispersant, at least one alcohol and the balance water.

19. The method of claim 18, wherein the at least one dispersant is selected from the group consisting of cationic dispersants, nonionic dispersants and anionic dispersants.

20. The method of claim 18, wherein the at least one alcohol is a polyhydric alcohol.

21. The method of claim 20, wherein the polyhydric alcohol is selected from the group consisting of butylene glycol and caprylyl alcohol.

22. A method of producing an antimicrobial carbon black dispersion, comprising feeding into a dispersal apparatus a composition comprising about 20% to about 50% by weight carbon black about 2% to about 40% by weight cationic block copolymer, about 40% by weight nonionic surfactant, at least about 10% polyhydric alcohol, optionally about 1% to about 20% by weight hydrophilic nonionic polymer, and about 30% to about 70% water; and using the dispersal apparatus to produce the antimicrobial carbon black dispersion from the composition.

23. The method of claim 22, wherein the composition comprises about 25% by weight carbon black, about 3% by weight cationic block copolymer, about 3% nonionic surfactant, about 11% by weight polyhydric alcohol, about 2% hydrophilic nonionic polymer, and about 56% by weight water.

24. The method of claim 22 wherein the dispersal apparatus is selected from the group consisting of high speed mixers, media mills, bead mills, ultrasonic mills and ultra-turrax.

25. The method of claim 24, wherein the dispersal apparatus is a media mill.

26. The method of claim 22, wherein the cationic block copolymer is alkyl acrylate and quaternized aminoalkyl acrylate.

27. The method of claim 22, wherein the nonionic surfactant is selected from the group consisting of acetylated lanolin alcohol, polysorbated 80, cetyl acetate, polyoxyethylene acrylic acid, amine oxides, alkanolamides, derivatives of carbohydrates, ethylene oxide/propylene oxide copolymers, ethoxylates of fatty acids, sorbitan derivatives, ethylene glycol esters, propylene glycolesters, glycerine esters and polyglycerine esters, alkylamines and alkyl-imidazolines and combinations thereof.

28. The method of claim 27, wherein the nonionic surfactant is selected from the group consisting of acetylated lanolin alcohol, polysorbate 80 and cetyl acetate.

29. The method of claim 22, wherein the hydrophilic nonionic polymer is selected from the group consisting of poly(N-vinylpyrrolidone), quaternary ammonium compounds, amine salts, imidazolium salts and combinations thereof.

30. The method of claim 29, wherein the hydrophilic nonionic polymer is poly(N-vinylpyrrolidone).

31. The method of claim 22, wherein the polyhydric alcohol is selected from the group consisting of butylene glycol, caprylyl glycol, propylene glycol, dipropylene glycol, tripropylene glycol, propylene carbonate and combinations thereof.

32. The method of claim 31, wherein the polyhydric alcohol is a mixture of butylene glycol and caprylyl glycol.

33. The method of claim 32, wherein the polyhydric alcohol is butylene glycol.

34. The method of claim 22, wherein the ratio of cationic block copolymer to nonionic surfactant is between about 1:5 and 5:1.

35. The method of claim 22, wherein the specific surface area of the carbon black is between about 100 to about 400 m²/gram.

36. The method of claim 35, wherein the specific surface area of the carbon black is between about 200 to about 260 m²/gram.

37. A method of using the antimicrobial carbon black dispersion of claim 1, comprising producing products containing the antimicrobial carbon black dispersion.

38. The method of claim 37, wherein the products are cosmetic products.

39. A method of using the antimicrobial carbon black dispersion of claim 5, comprising producing products containing the antimicrobial carbon black dispersion.

40. The method of claim 39, wherein the products are cosmetic products.

41. A cosmetic product made by the method of claim 38, made by mixing an antimicrobial carbon black dispersion with about 20% to about 50% by weight carbon black, about 2% to about 40% by weight cationic block copolymer, about 2% to about 40% by weight nonionic surfactant, at least about 10% polyhydric alcohol, optionally about 1% to about 20% by weight hydrophilic nonionic polymer, and about 30% to about 70% water.

42. A product as in claim 41, wherein the product is formulated to be solid and may be abraded onto an applicator for rubbing onto the skin.

43. A product as in claim 41, wherein the product is formulated to be liquid and may be coated onto an applicator for painting on the skin.
